(19) United States
(12) Patent Application Publication
(10) Pub. No.: US 2004/0147766 A1
(43) Pub. Date: Jul. 29, 2004

(54) Intermediates for producing spinosyns

(76) Inventors: Marc Stadler, Wuppertal (DE); Anke Mayer-Bartschmid, Wulfrath (DE); Stephan Seip, Schwelm (DE); Robert Velten, Koln (DE); Peter Jeschke, Bergisch Gladbach (DE); Karlheinz Weber, Wuppertal (DE)

Correspondence Address: Bayer CropScience, Patent Department, 100 Bayer Road, Pittsburgh, PA, 15205-9741, US

(21) Appl. No.: 10/473,318

(22) PCT Filed: Mar. 19, 2002
(86) PCT No.: PCT/EP02/02999

(30) Foreign Application Priority Data
Mar. 29, 2001 (DE) 101 15 407.0
May 2, 2001 (DE) 101 21 313.1

(51) Int. Cl. C07D 313/20

(52) U.S. Cl. 549/268

(57) ABSTRACT

The present invention relates to intermediates for preparing spinosyns, to a variety of processes for their preparation, and to the use of these intermediates for preparing spinosyn derivatives.

The present invention relates to intermediates for preparing spinosyns, to a variety of processes for their preparation, and to the use of these intermediates for preparing spinosyn derivatives.

The spinosyns are known compounds. Spinosyns are fermentation products produced by cultures of the actinomycete Saccharopolyspora spinosa. Natural spinosyns consist of a tetracyclic polyketide skeleton (aglycon) with a 12-membered macrolide ring and a 5,6,5-cis-anti-trans tricycle, and of a D-forosamine and a 2,3,4-tri-O-methyl-L-rhamnose sugar moiety (Kirst et al. (1991), Tetrahedron Letters, 32:4839). Over 20 different natural spinosyns, what is known as the A83543 complex, have been described to date (cf. WO 97/00265, WO 94/20518 and WO 93/09126). These compounds differ in the substitution of one or some methyl groups at the tetracyclic skeleton, at the forosamine or at the trimethyl rhamnose sugar moiety. A 17-pseudoaglycon which lacks the forosamine sugar moiety, has also been isolated from culture broths of S. spinosa.

The main components of the A 83543 complex formed by S. spinosa of the variants spinosyn A and spinosyn D, which constitute essential components of the product spinosad (cf. Pesticide Manual, British Crop Protection Council, 11^{th} Ed., 1997, page 1272 and Dow Elanco trade magazine Down to Earth, Vol. 52, NO: 1, 1997 and the literature cited therein).

If the compounds lack the amino sugar, they are referred to as spinosyn A, D, etc.—17-pseudoaglycon, when they lack the neutral sugar, they are referred to as spinosyn A, D, etc.—9-pseudoaglycon. Spinosyns without the two sugar residues are referred to as spinosyn aglycon.

Spinosyns are suitable for controlling arachnids, nematodes and insects, in particular Lepidoptera and Diptera. It can be expected that plant pests which are currently controlled by spinosyns will be able to generate a resistance to these commercially available active substances. It is therefore important to prepare new biologically active spinosyn derivatives which are capable of replacing spinosyns currently being used for controlling pests. Synthetic approaches in the preparation of spinosyn derivatives were described by Martynow, J. G. and Kirst, H. A. in J. Org. Chem. 1994, 59, 1548. The 9,17-diketone of the spinosyn aglycon and the 17-keto derivative of the spinosyn aglycon are mentioned in this publication. The spinosyn-17-pseudoaglycon and spinosyn-9-pseudoaglycon are disclosed in WO 97/00265 and U.S. Pat. No. 6,001,981. These documents also describe the preparation of further derivatives which can be obtained by semisynthetic methods starting from natural products. The spinosyn-9-pseudoaglycon, which is oxidized at position C9, is known as insecticidal compound which has been generated by chemical derivatization. The 9-keto-spinosyn aglycon is new with regard to the prior art.

It is the aim of the present invention to provide novel intermediates which are suitable for the preparation of spinosyn derivatives.

This aim was achieved by providing compounds of the formula (I)

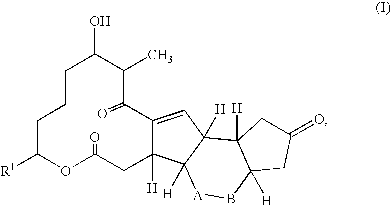

in which

R¹ represents methyl or ethyl, and

A-B represents one of the following groups: —HC═CH—, —HC═C(CH₃)—, —H₂C—CH₂—, —H₂C—CH(CH₃)—.

R¹ preferably represents ethyl.

A-B preferably represents the group —HC═CH—.

The compounds of the formula (I) according to the invention can exist in the form of stereomers which behave either like image and mirror image (enantiomers) or which do not behave like image and mirror image (diasteromers). The invention relates both to the enantiomers or diastereomers or their respective mixtures. The racemic forms, like diastereomers, can be resolved into the stereoisomerically uniform components in the known manner. If appropriate, the isomers can be converted into each other by methods known per se.

The present invention also relates to chemical and biochemical/microbiological methods for the preparation of the abovementioned compounds of the general formula (I).

The compounds of the formula (I) are obtained when compounds of the formula (II)

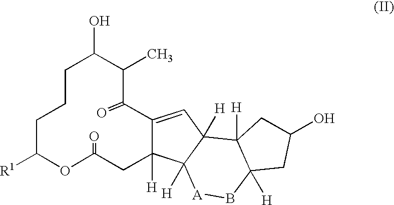

in which R¹ and A-B have the abovementioned meanings,

are reacted with an oxidant, if appropriate in the presence of a diluent.

The spinosyn aglycon which can be used as starting compound for the method according to the invention is known and can be prepared by the method described in WO 01/16303. Analogously, the starting compounds which can be used for the method according to the invention can be obtained starting from the corresponding natural spinosyns.

Many different oxidants are known for the oxidation of alcoholic groups (cf., for example, oxidants in: Organic Synthesis by Oxidation with Metal Compounds; Mijs, de Jonge; Plenum: New York, 1986; Manganese Compounds as Oxidizing Agents in Organic Chemistry; Arndt, Open Court Publishing Company: La Salle, Ill., 1981; The Oxidation of Organic Compounds by Permanganate Ion and Hexavalent Chromium; Lee, Open Court Publishing Company: La Salle, Ill., 1980). Accordingly, an oxidation can be carried out for example in the presence of permanganates such as potassium permanganate, halogens such as chlorine or bromine, metal oxides such as manganese dioxide or ruthenium tetraoxide, and the like.

Many different oxidants are also described in the literature specifically just for the oxidation of secondary alcohols, such as, for example, the use of acidic dichromates (cf. Chromium Oxidations in Organic Chemistry; Cainelli, Cardillo, Springer: New York, 1984; Reagents for Organic Synthesis; Fieser, Vol. 1, Wiley: New York, 1967, pp. 142-147, 1059-1064 and further volumes in this series). A solution of chromic acid and sulfuric acid in water is known as Jones's reagent (Bowden et al., (1946), J. Chem. Soc.: 39; Bowers et al. (1953), J. Chem. Soc.: 2548). Three other chromium(VI) reagents (see communication of a comparative study of Jones's, Collins's and Corey's reagents in Warrener et al. (1978), Aust. J. Chem., 31: 1113) are also used, as is known, for example dipyridin/chromium(VI) oxide (Collins's reagent) (cf., for example, Collins et al. (1968), Tetrahedron Lett.: 3363), pyridinium chlorochromate (Corey's reagent) (cf., Review: Luzzio and Guziec (1988), Org. Prep. Proced. Int., 20: 533-584) and pyridinium dichromate (cf., Coates (1969), Corrigan Chem. Ind. (London): 1594; Corey, Schmidt (1979), Tetrahedron Lett.: 399). Others which are known for acid-sensitive substrates are, for example, chromium(VI) oxide in hexamethylphosphoric triamide (HMPA) (cf., Cardillo et al. (1976), Synthesis: 394), a chromium(VI) oxide/pyridine complex (cf., Poos et al. (1953), J. Am. Chem. Soc., 75: 422) or trimethylsilyl chromate (Moiseenkov et al. (1987), J. Org. Chem. USSR, 23: 1646). Sodium hypochlorite in acetic acid is mentioned for the oxidation of secondary alcohols in large amounts (cf., Stevens et al. (1980), J. Org. Chem., 45: 2030; Schneider et al. (1982), J. Org. Chem., 47: 364). However, the oxidants may also be present in polymer-bound form (cf., Review: McKillop, Young (1979), Synthesis: 401422). Both chromic acids and permanganates were used in this manner as oxidants. Also known are a large number of phase transfer reactions involving permanganates (cf., Review: Lee, in Trahanovsky, Ref. 2, pt. D, S. 147-206), chromic acids (Hutchins et al. (1977), Tetrahedron Lett.: 4167; Landini et al. (1979), Synthesis: 134) and ruthenium tetroxide (Morris, Kiely J. Org. Chem. (1987), 52: 1149). Even ultrasound-induced oxidation reactions are feasible—thus, the use of potassium permanganate is mentioned (Yamawaki et al. (1983), Chem. Lett.: 379).

In addition, most of the oxidants which are capable of oxidizing primary alcohols to aldehydes are suitable, as they are for the corresponding oxidation of secondary alcohols. Examples of such oxidants for primary alcohols are pyridinium dichromate, tetrapropylammonium perruthenate (Pr₄N RuO₄⁻), cerium ammonium nitrate (CAN), silver carbonate on Celite (Fetizon et al. (1968), Acad. Sci., Ser. C, 267: 900), Na₂Cr₂O₇ in water (Lee et al. (1970), J. Org. Chem., 35: 3589), lead tetraacetate/pyridine, benzoyl peroxide/nickel dibromide or dimethyl sulfoxide in the presence of oxalyl chloride (Swern oxidation), copper(II) sulfate pentahydrate in pyridine, copper(II) acetate in 70% acetic acid, iron chloride in water, chromium(VI) oxide in glacial acetic acid or dichromium trioxide in pyridine. The reagents which are capable of specifically oxidizing a secondary hydroxyl group, even in the presence of a primary hydroxyl group, include, for example, hydrogen peroxides/ammonium molybdate (Trost et al. (1984), Isr. J. Chem., 24: 134), sodium borate (NaBrO₃)-CAN (Tomioka et al., Tetrahedron Lett. 23: 539). N-halosuccinimides (halo=chloro, bromo, iodo) can be employed as oxidants for hydroxyl groups even in the presence of other groups capable of being oxidized (variant of Corey and Khim (1972), J. Am. Chem., Soc., 94:7586; Review: Filler (1963), Chem. Rev., 63: 21-43, p. 22-28). For example, the combination of N-iodosuccinimide and tetrabutyl ammonium iodide is suitable for the oxidation of secondary alcohols in high yields (Hanessian et al. (1981), Synthesis: 394).

Further known oxidation methods also include oxidative dehydrogenation, for example in the presence of catalysts such as silver or copper catalysts (M. Muhler in: Handbook of Heterogenous Catalysis, VCH, Weinheim, 1997). Other mild catalytic oxidative processes using platinum/carbon or palladium/carbon catalysts, which even permit the oxidation of classes of sensitive substances, for example carbohydrates (M. Besson et al. (1995), J. Catal. 152: 116-122) or steroids (T. Akihisa et al. (1986), Bull. Chem. Soc. Jpn. 59: 680-685), are known. An example of an efficient commercial catalyst for the oxidation is the inorganic TS-1 catalyst (oxide titanium silicalite), which makes possible the catalytic oxidation of primary and secondary alcohols in aqueous hydrogen peroxide (30% w/w) (R. Murugawel et al. (1997), Angew. Chem. Int. Ed. Engl., 36: 477-479).

Oxidants which are preferably employed are N-halosuccinimides, in particular N-chlorosuccinimide, in the presence of dimethyl sulfide (Swern oxidation), or pyridinium dichromate.

The process according to the invention for the preparation of the new compounds is preferably carried out using diluents. Diluents are preferably employed in such an amount that the reaction mixture remains readily stirrable during all of the process.

Depending on the abovementioned oxidant, suitable diluents other than water or aqueous hydrogen peroxides are acidic diluents such as, for example, concentrated or partially diluted acetic acid, and basic diluents, such as, for example, pyridine.

Furthermore, virtually all of the inert organic solvents are suitable. These include, in particular, aliphatic, alicyclic or aromatic, optionally halogenated hydrocarbons such as, for example, benzine, benzene, toluene, xylene, anisole, chlorobenzene, dichlorobenzene, petroleum ether, hexane, cyclohexane, dichloromethane, 1,2-dichloroethane, chloroform, carbon tetrachloride, ethers, such as diethyl ether, dioxane, tetrahydrofuran, ethylene glycol dimethyl ether or ethylene glycol diethyl ether, ketones such as acetone or butanone, nitriles such as acetonitrile, propionitrile, amides such as dimethylformamide, dimethylacetamide, N-methylformanilide, N-methylpyrrolidone or hexamethylphosphoric triamide, esters such as ethyl acetate, sulfoxides such as dimethyl sulfoxide, or sulfolane.

Naturally, the process according to the invention can also be carried out in mixtures of the abovementioned solvents.

Dichloromethane is particularly preferably employed as the diluent.

Moreover, the reaction in accordance with the process according to the invention is preferably carried out under inert gas.

When carrying out the process according to the invention, the reaction temperatures can be varied within a substantial range. In general, the process is carried out at temperatures of between −100° C. and +150° C., preferably at temperatures between −20° C. and +50° C.

In general, the process according to the invention is carried out under atmospheric pressure. However, it is also possible to carry out the process under elevated or reduced pressure.

To carry out the process according to the invention, the starting compounds required in each case are generally employed in approximately equimolar amounts. However, it is also possible to employ substoichiometric amounts of the oxidant.

Work-up is carried out by customary methods.

The new compounds of the general formula (I) can also be carried out by bioconversion, starting from compounds of the general formula (II).

Selective and/or stereospecific oxidations of hydroxyl groups of natural products and of synthetic compounds by means of bioconversion using microorganisms or their enzymes are described in the literature. In particular, cells and/or enzymes of Actinomycetes (Nocardia, Streptomyces) and other Gram-positive (Bacillus, Clostridium) or Gram-negative bacteria have been used for the specific oxidation of steroid compounds where limitations apply with regard to the chemical synthesis. Examples concerning in particular enzyme classes such as hydroxysteroid dehydrogenases or cholesterol oxidazes are listed hereinbelow:

| Organism/Enzyme | Chemical compound class | Reference |
|---|---|---|
| Multi-enzyme system | D-ketoursocholic acid | Bovara, R. et al. |
| Clostridium absonum/Bacillus | (steroid) | (1996): Biotechnology |
| megaterium/Proteus; 7-alpha- | | Lett. 19, 305-308 |
| hydroxysteroid-dehydrogenase | | |
| (HSDH) + 12-alpha-HSDH and | | |
| other enzymes | | |
| Bacillus stearothermophilus | Bicyclic derivatives of | Giovannini et al. |
| cells | octenoic acid | (1996): Tetrahedron |
| | | 52, 1669-1676 |
| Streptomyces sp. | delta-4-cholestenone | Lee & Bielmann |
| Cholesterol oxidase | (steroid) | (1988): Tetrahedron |
| | | 44, 1135-1139 |
| Multi-enzyme system | Cholic acid derivatives | Riva, S. et al. (1986): |
| Clostridium absonum/Proteus; 3- | (steroids) | J. Org Chem. 51, |
| alpha-hydroxysteroid- | | 2902-2906 |
| dehydrogenase and other enzymes | | |
| E. coli; 7-alpha-hydroxysteroid- | Cholic acid derivatives | Bovara et al. (1993): J. |
| dehydrogenase (HSDH) | (steroids) | Org. Chem. 58, |
| | | 499-501 |
| Brevibacterium sp. | 7-beta- | Labaree et al. (1997): |
| Cholesterol oxidase | hydroxytestosterone | Steroids 62, 482-486 |
| | (steroid) | |
| Nocardia rhodochrous; cells and | Diosgenone (steroid) | Saunders et al. (1986) |
| cholesterol oxidase | | Enzyme Microb. |
| | | Technol. 9, 549-555 |

In accordance with the invention, compounds of the formula (II)

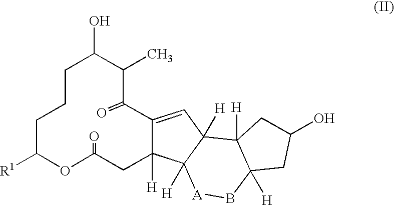

where R¹ and A-B have the abovementioned meanings,

can be brought into contact with a microorganism in an aqueous nutrient medium under aerobic conditions, followed by isolation of compounds of the formula (I).

Instead of the microorganisms, it is also possible to use enzyme extracts and purified enzymes which can be produced by customary processes starting from these microorganisms, may also be used, if appropriate after addition, or with regeneration, of the cofactors required.

A microorganism of the genus Bacillus or from the group of the Actinomycetes, in particular the genus Streptomyces, or a fungus, in particular from the class of the Zygomycetes, preferably the genera Zygorhynchus or Mucor, or enzyme extracts or purified enzymes produced using the above starting materials, are preferably used for the process according to the invention.

A strain of the genus Bacillus simplex, Bacillus megaterium, Streptomyces argillaceus, Streptomyces scabies, Streptomyces mirabilis, Streptomyces pseudovenecuelae, Zygorhynchus moelleri or Mucor circinelloides is especially preferably employed in the process according to the invention.

A strain with the characterizing traits of the following strains is very especially preferably used for the process according to the invention:

| Name | Deposit No. |
|---|---|
| Streptomyces argillaceus | DSM 14030 |
| Streptomyces scabies | DSM 14029 |
| Bacillus megaterium | DSM 333 |
| Bacillus megaterium | DSM 339 |
| Bacillus simplex | DSM 14028 |
| Streptomyces spec. | DSM 14077 |
| Streptomyces mirabilis | DSM 14078 |
| Streptomyces pseudovenecuelae | DSM 14079 |
| Zygorhynchus moelleri | DSM 14198 |
| Mucor circinelloides | DSM 14199 |

The strains mentioned in the table have been deposited at the Deutsche Sammlung von Mikroorganismen und Zellkulturen GmbH [German Collection of Microorganisms and Cell Cultures] (DSMZ), Mascheroder Weg 1b, D-38124 Brunswick, Germany, in compliance with the provisions of the Budapest treaty.

The strains are described in further detail in Example 9. Not only the deposited strains as such, but also their mutants, may be used as long as these mutants have the characterizing traits of the strains deposited. This means that the mutants must retain the capability of carrying out the bioconversion according to the invention.

Preferably, the aqueous nutrient medium contains an assimilable carbon source and an assimilable nitrogen source.

The compounds of the formula (I) are produced, for example, when a strain from among the species mentioned in the table is fermented in an aqueous nutrient medium under aerobic conditions in the presence of compounds of the formula (II).

Typically, the microorganisms are fermented in a nutrient medium containing a carbon source and, if appropriate, a proteinaceous material. Preferred carbon sources encompass glucose, brown sugar, sucrose, glycerol, starch, corn starch, lactose, dextrin, molasses and the like. Preferred nitrogen sources encompass cotton seed meal, yeast, autolyzed baker's yeast, solid milk constituents, soya meal, corn meal, casein hydrolysates (pancreatic or papainic), solid distillation components, broths of animal peptone, meat and bone fragments, and the like. Preferably, combinations of these carbon and nitrogen sources are used. Trace elements such as, for example, zinc, magnesium, manganese, cobalt, iron and the like need not be added to the fermentation medium as long as tap water and unpurified constituents are being used as components of the medium.

The production of the compounds of the general formula (I) can be induced at any temperature which ensures sufficient growth of the microorganisms. The temperature is preferably between 21° C. and 32° C., especially preferably approximately 28° C.

In general, optimal production of the compounds of the formula (I) is achieved within 1 to 10 days after the compounds of the formula (II) have been added to the culture, preferably in approximately 2 to 6 days. Normally, the fermentation broth remains weakly basic during the fermentation (pH 7.4 to pH 8.0). The final pH depends partly on the buffer which is optionally used and partly on the initial pH of the culture medium. Preferably, the pH is brought to approximately 6.5 to 7.5 prior to sterilization, especially preferably to pH 7.2.

Production of the compounds according to the invention takes place in shake flasks or else in stirred fermenters. If culturing takes place in shake flasks or in large reactors and tanks, it is preferred to use the vegetative form of the microorganisms for inoculation instead of the spore form in order to avoid a pronounced lag phase in the production of the metabolites and thus inefficient use of the equipment. Accordingly, it is advantageous to prepare a vegetative inoculum in aqueous nutrient medium by inoculating this medium with an aliquot of a bottom or slant culture.

After fresh, active, vegetative inoculum has been prepared in this manner, it is transferred aseptically into other shake flasks or other suitable equipment for the fermentation of the microorganisms. The medium in which the vegetative inoculum is prepared can be identical with, or different from, the medium which is used for the production of the compounds according to the invention as long as it ensures sufficient growth of the microorganisms.

In general, the production of the compounds according to the invention is performed in stirred fermenters under aerobic conditions using the above-mentioned microorganisms. However, the production is independent of the fermenters and starter cultures used. The compounds according to the invention may also be obtained via shake cultures. Vegetative inoculum is preferably used for large-scale fermentations. The vegetative inoculum is prepared by inoculating a small volume of the culture medium with the spore form, mycelial fragments or a lyophilized pellet of the microorganism. The vegetative inoculum is then transferred into a fermentation reactor in which the compounds according to the invention are produced in optimal yield after a suitable incubation period in the presence of the compounds of the general formula (II). Usually, sterile air is passed through the culture medium in the case of an aerobic submerged fermentation process. The volume of air used for efficient growth of the microorganisms is in the range of from approximately 0.25 to approximately 0.5 volumes of air per volume of culture medium per minute (vvm). An optimal ratio in a 10 l reactor is approximately 0.3 vvm with agitation, which is generated by a conventional propeller which rotates at approximately 200-500 rpm, preferably 300 rpm. If foaming is a problem, the addition, for the fermentation medium, of a small amount such as, for example, 1 ml/l, of an antifoam such as silicone is necessary.

Preferred fermentation conditions and media are described in the examples.

In general, the biotransformation of the compounds of the formula (II) to the compounds according to the invention starts after approximately 48 hours and takes place for at least 6 days during the fermentation period. Peak production is, reached between approximately 5 to 7 days of the fermentation time, if using Bacillus strains as early as after 3-4 days.

The compounds according to the invention in the form of the biotransformation product can be isolated from the fermentation medium by customary processes.

The compounds according to the invention are mainly present in the biomass of the fermented microorganisms, but may also occur in small amounts in the culture filtrate of the fermentation broth. The culture broth can be removed simply by filtering through a filter press.

A variety of methods may be employed in the isolation of the compounds according to the invention from the fermentation broth and their purification, such as, for example, chromatographic adsorption methods (for example column chromatography, liquid-liquid partitioning chromatography, gel permeation chromatography) followed by elution with a suitable solvent, and crystallization of solvents, and combinations of these. In a preferred purification method, the compounds according to the invention are extracted from the biomass, from the mycelia or from extracts of the supernatant. These latter can be generated by using adsorption resins, such as, for example, XAD, HP20 or Lewapol. Column-chromatographic methods, preferably over silica gel or modified silica gels, are used for the initial purification. A final purification of the compounds according to the invention is preferably achieved by preparative high-performance liquid chromatography (HPLC).

The compounds according to the invention can be prepared for the preparation of biologically active, in particular insecticidal, spinosyn derivatives.

If, for example, the glycosidation of the compound of the formula (Ia) according to the invention involves the use of an activated forosamine (D-forosamine: cf. EP-A 0 375 316) as amino sugar of the formula (III) in which LG can represent a known, suitable leaving group (LG) such as, for example, bromo or 2,2,2-trichloroethaneimidate (=trichloroacetimidate), the 9-keto-spinosyn A-9-pseudoaglycon of the formula (IVa), which is known from WO 97/00265 and U.S. Pat. No. 6,001,981 is obtained (cf. Scheme 1).

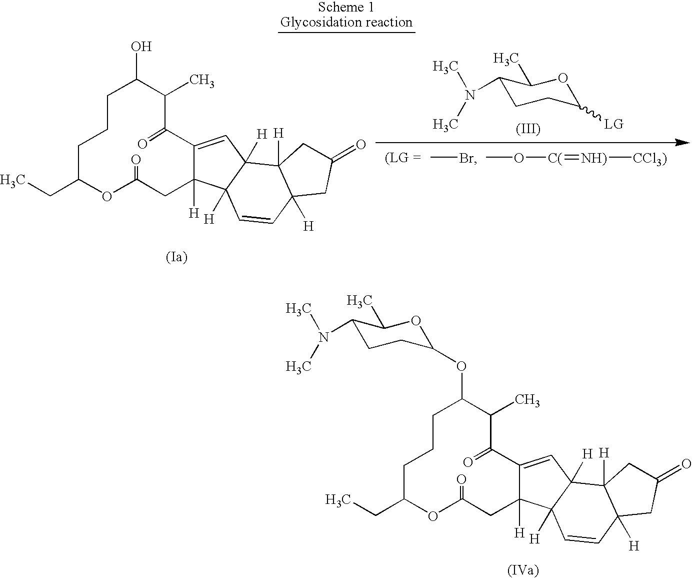

The synthesis of an activated forosamine as amino sugar of the formula (III), for example of the α-D-forosaminyl bromide hydrobromide or of the α-L-N-Fmoc-forosaminyl bromide (LG=bromo), and its glycosidation reaction, is known from WO 97/00265 and U.S. Pat. No. 6,001,981 or else D. A. Evans et al. (1993), J. Am. Chem. Soc. 115: 4497-4513. Furthermore, the synthesis of an activated amino sugar of the formula (III), for example of the 1-(2,2,2-trichloroethaneimidate)-4,6-dideoxy-4-(dimethylamino)-5-C-methyl-β-D-ribo-hexopyranose-2,3-diacetate (LG=2,2,2-trichloroethaneimidate), and its stereoselective glycosidation reaction, is furthermore known (cf. I. Sato et al. (1999), Chem. Lett. 9: 867-868; cf. additionally also the use of trichloroacetimidates in the preparation of glucospingolipids: G. R. Duffin et al. (2000), J. Chem. Soc., Perkin Trans. 1: 2237-2242).

The biological activity of the 9-keto-spinosyn A-9-pseudoaglycon of the formula (IVa) against Stomoxys calcitrans (stable fly) and Phormia regina (blow fly) is already described in WO 97/00265 and U.S. Pat. No. 6,001,981.

Moreover, WO 97/00265 and U.S. Pat. No. 6,001,981 disclose two subsequent reactions (route a and b) with the 9-keto-spinosyn A-9-pseudoaglycon of the formula (IVa) in which the reactivity of the carbonyl group in the 9-position is exploited (cf. scheme 2).

For example, it is possible to convert the 9-keto-spinosyn A-9-pseudoaglycon of the formula (IVa) with the Grignard reagent methylmagnesium chloride (V) to give a mixture of (9S)- and (9R)-9-methyl-spinosyn A-9-pseudoaglycon of the formula (VIa) and (VIb), which mixture can be resolved by chromatography (route a). The reaction of the 9-keto-spinosyn A-9-pseudoaglycon of the formula (IVa) with morpholine (VII) in the presence of sodium cyano borohydride with formation of the 9-deoxy-9-(N-morpholinyl)-spinosyn A-9-pseudoaglycon of the formula (VI) is illustrated (cf. Scheme 2, route b) in a further known use example.

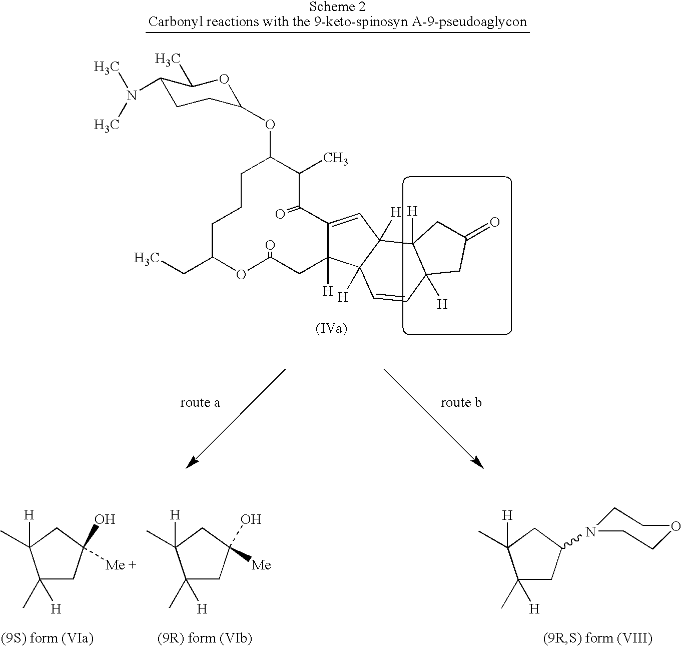

Route a: MeMgCl (V), tetrahydrofuran

Route b: Morpholine(VII), methanol, sodium cyanoborohydride

A biological, in particular insecticidal, activity of the (9S)- and (9R)-9-methyl-spinosyn A-9-pseudoaglycon of the formula (VIa) and (VIb) against Aphis gossipii (cotton aphid), Heliothis virescens (tobacco budworm) and Stomoxys calcitrans (stable fly) has already been described in WO 97/00265 and U.S. Pat. No. 6,001,981. A corresponding biological, in particular insecticidal, activity against Stomoxys calcitrans (stable fly) has also been shown in WO 97/00265 and U.S. Pat. No. 6,001,981 for the 9-deoxy-9-(N-morpholinyl)-spinosyn A-9-pseudoaglycon of the formula (VIII).

### EXAMPLES

### Example 1

Production of the Spinosyn Aglycon from Tracer®

The compound of the formula (II), where R¹ represents ethyl and A-B represents the group —HC═CH— (hereinbelow referred to as compound (2)) was produced as described in WO 01/16303.

### Example 1A

Production of the 5,6-dihydrospinosyn-aglycon from Tracer®

The compound of the formula (II), in which R¹ represents ethyl and A-B represents the group —H₂C—CH₂— (5,6-dihydrospinosyn aglycon, herein referred to as compound (4)) was produced analogously to WO 01/16303 from 5,6-dihydrospinosyn A. The synthesis of 5,6-dihydrospinosyn A from Spinosyn A is disclosed in WO 97/00265 and U.S. Pat. No. 6,001,981. Spinosyn A, in turn, was produced from Tracer® as described in WO 01/16303.

### Example 2

Strains Used

Table: strains which are capable of the biotransformation of compound (2) into the corresponding 9-keto derivative (hereinbelow referred to as compound (1)):

| Name | Internal name | Deposit No. |
|---|---|---|
| Streptomyces argillaceus | ATCC 12956 | DSM 14030 |
| Streptomyces scabies | BS 2134 | DSM 14029 |
| Bacillus megaterium | DSM 333 | DSM 333 |
| Bacillus megaterium | DSM 339 | DSM 339 |
| Bacillus simplex | DSM 1318 | DSM 14028 |
| Streptomyces spec. | BA 312 | DSM 14077 |
| Streptomyces mirabilis | BA 579 | DSM 14078 |
| Streptomyces pseudovenecuelae | WS 2199 | DSM 14079 |
| Zygorhynchus moelleri | WP0796 | DSM 14198 |
| Mucor circinelloides | WP0799 | DSM 14199 |

ATCC = American Type Culture Collection, Rockville, MD.
DSM = Deutsche Sammlung von Mikroorganismen und Zellkulturen [German Collection of Microorganisms and Cell Cultures] Brunswick, Germany

### Example 3

Biotransformation with Streptomyces argillaceus DSM 14030, Streptomyces scabies DSM 14029, Streptomyces spec. DSM 14077, Streptomyces mirabilis DSM 14078, Streptomyces pseudovenecuelae DSM 14079, Zygorhynchus moelleri DSM 14198 or Mucor circinelloides DSM 14 199

Example of a protocol of the biotransformation for the production of compound (1) from compound (2).

### Example 3A

Preparation of the Precultures of Streptomyces argillaceus DSM 14030, Streptomyces scabies DSM 14029, Streptomyces spec. DSM 14077, Streptomyces mirabilis DSM 14078, Streptomyces pseudovenecuelae DSM 14079, Zygorhynchus moelleri DSM 14198 or Mucor circinelloides DSM 14199

To prepare the precultures, the strains Streptomyces argillaceus DSM 14030, Streptomyces scabies DSM 14029, Streptomyces spec. DSM 14077, Streptomyces mirabilis DSM 14078, Streptomyces pseudovenecuelae DSM 14079, Zygorhynchus moelleri DSM 14198 or Mucor circinelloides DSM 14199 were grown in the following media: medium 1: yeast malt medium: D-glucose 0.4%, yeast extract 0.4%, malt extract 1.0%, tap water to 1 liter (pH brought to 6.5 in the case of the fungi, using aqueous HCl, and to 7.2 in the case of the bacteria, using aqueous NaOH) or TSB medium (medium 2): Trypticase Soy Broth (Difco) (30 g/l), tapwater to 1 liter. The pH value of the medium was brought to 7.2 using aqueous NaOH. All of the media were sterilized for 20 minutes at 121° C. and a superatmospheric pressure of 1.1 bar.

2 ml portions of a mycelial suspension in 50% glycerol were used as inoculum for 2×150 ml medium 1 in a 1000 ml Erlenmeyer flask and incubated for 72 hours at 240 rotations per minute (rpm) on an orbital shaker. These cultures were either used for the preparation of fresh glycerol preserves, which were stored at −20° C., or they acted as inoculum for the production cultures (see 3b).

### Example 3B

Preparation of the Production Cultures of Streptomyces argillaceus DSM 14030, Streptomyces scabies DSM 14029, Streptomyces spec. DSM 14077, Streptomyces mirabilis DSM 14078, Streptomyces pseudovenecuelae DSM 14079, Zygorhynchus moelleri WP0796 or Mucor circinelloides DSM 14199

To prepare the production cultures, 2 ml portions of a preculture as described under 3a) were employed as inoculum for 100×150 ml medium GS (glucose (20 g/l), soya meal (20 g/l), starch (20 g/l), NaCl (2.5 g/l), CaCO₃ (5 g/l), MgSO₄×6H₂O (0.5 g/l), KH₂PO₄ (0.25 g/l). Prior to the inoculation, the pH value was brought to 6.8 using KOH, and the media were sterilized for 20 minutes at 121° C. and superatmospheric pressure of 1.1 bar. Compound (2) was added at a final concentration of 5-500 mg/l medium in the form of a methanolic solution (100 mg/ml methanol), either at the beginning of the fermentation or during the fermentation at a desired point in time up to a fermentation time of 160 hours. The biotransformation was stopped after 240 hours. 50 ml samples taken daily under sterile conditions which were analyzed with the aid of analytical HPLC were used to monitor the biotransformation process. Under these conditions, conversion rates of up to 100% of compound (2) into compound (1) were achieved, preferably using S. argillaceus DSM 14030.

### Example 4

Biotransformation with Bacillus simplex DSM 14028, B. megaterium DSM 333 and B. megaterium DSM 339

Example of a protocol of the biotransformation for the production of compound (1) from compound (2).

### Example 4A

Preparation of the Primary Precultures of Bacillus simplex DSM 14028, B. megaterium DSM 333 and B. megaterium DSM 339

To prepare the primary precultures, Bacillus simplex DSM 14028, B. megaterium DSM 333 and B. megaterium DSM 339 were grown in the following medium: TSB medium (medium 2): Trypticase Soy Broth (Difco) (30 g/l), tapwater to 1 liter. The medium was sterilized for 20 minutes at 121° C. and a superatmospheric pressure of 1.1 bar.

2 ml of a bacterial suspension in 50% glycerol were used as inoculum for 150 ml of medium 2 in a 1000 ml Erlenmeyer flask and incubated for 48 hours at 240 rotations per minute (rpm) on an orbital shaker. These cultures were either used for the preparation of fresh glycerol preserves, which were stored at −20° C., or they acted as inoculum for the production cultures (see 4b).

### Example 4B

Production Culture (10-Liter Scale)

To prepare the production culture, 1×150 ml of a preculture described as under 4a) was employed as inoculum for 10 liters of medium 3 (LB Broth Medium, Sigma). Prior to the inoculation, 1 ml of antifoam SAG 5693 (Union Carbide, USA) was added per liter of medium, and the medium was steam-sterilized for 30 minutes at 1.1 bar. This production culture was incubated for 90 hours at 28° C. in a 10 l Giovanola stirred fermenter (blade stirrer) at a stirring speed of 300 rpm and an aeration rate of 0.3 vvm. Compound (2) was added at the beginning of the fermentation in a final concentration of 50-250 mg/l medium in the form of a methanolic solution (2.5 g dissolved in 60 ml of methanol). Daily samples of 50 ml which were taken under sterile conditions and analyzed with the aid of analytical HPLC were used to monitor the biotransformation process. Under these conditions, conversion rates of up to 60% of compound (2) and compound (1) were achieved, preferably using B. simplex DSM14028.

### Example 5

Analytical HPLC

Analytical HPLC methods with UV/visual (HPLC-UV/Vis) and mass-spectrometric detection were employed during the biotransformation for detection purposes.

Prior to the HPLC analysis, the samples were dissolved in MeOH and filter-sterilized.

HPLC-MS were carried out using an HP1100 BPLC-System coupled with a Micromass-LCT mass spectrometer (Micromass, Manchester, Great Britain) in ESI+ mode. Mass spectra were recorded in the range between 200 and 1200. Compound (1) had the retention time of 4.08 minutes. The HP1100 system used operated with the following parameters: stationary phase: Waters Symmetry C18, 3.5 μm 2.1×50 mm column, mobile phase: gradient water(A), acetonitrile (B)+0.1% formic acid (0-1 minutes 100% A; 1-5 minutes linear gradient to 10% A/90% B ; 5-6 minutes 10% A/90% B, 6-6.10 minutes 90% B -100% B). Compound (1) eluted at 4.08 minutes. Detection was based on the protonated molecule ions (M+H)⁺.

HPLC-UV/Vis analyses were carried out with the aid of a Hewlett Packard Series 1100 analytical HPLC system (HP, Waldbronn, Germany), consisting of a G 1312A binary pump system, a G 1315A diode array detector, a G 1316A column thermostat system, a G 1322A degassing system and a G 1313A autoinjector. The mobile phase used was 0.01% H₃PO₄: acetonitrile (ACN) at a flow rate of 1 ml/minute, while a Merck (Darmstadt, Germany) Lichrospher RP 18 column (125×4 mm, particle size 5 μm) acted as the stationary phase. The samples were separated in a continuous linear gradient (0% ACN to 100% ACN in 10 minutes), followed by isocratic elution (5 minutes at 100% ACN). HPLC-UV chromatograms were recorded at 210 nm (detection of contaminants) and 254 nm (optimum detection in the UV maximum of the spinosyn derivatives) with a respective reference wavelength of 550 nm at a band width of 80 nm. Diode array detection in the range 210-600 nm gave the HPLC/UV/Vis spectra. Data were stored with the aid of the HP ChemStation Software. Compound (2) had a retention time of 7.85-7-9 minutes, while compound (1) eluted at a retention time of 8.21-8.27 minutes.

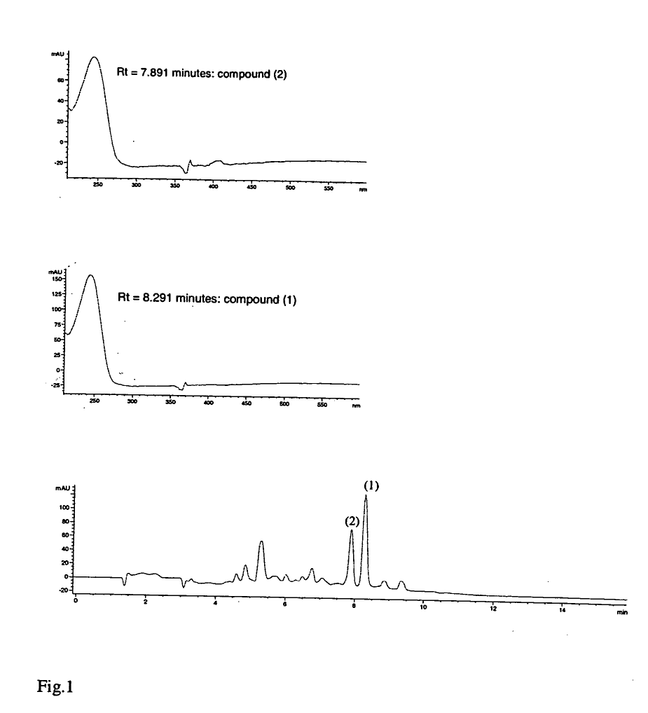

Internal and external standards of the pure substances were used for the analytical detection of compound (2) and compound (1) in fermentation samples, and detection was carried out at matching retention times in the two different analytical systems, including the respective HPLC-UV/Vis and HPLC-MS spectra. FIG. 1 shows an BPLC-UV chromatogram of the crude extract at 254 nm and HPLC-/UV spectra (diode array; Rt=retention times) of compound (2) and compound (1) from a fermentation sample of S. argillaceus (GS medium) after a fermentation time of 146 hours.

### Example 6A

Extraction and Preparative Purification of Compound (1) from Shake Cultures

Ten 150 ml cultures of strain (S. argillaceus DSM 14030) in 1000 ml Erlenmeyer flasks, which had been treated with 50 mg/l of compound (2), were harvested after 240 hours, and the culture broths were combined and freeze-dried. The residues obtained after freeze-drying were extracted twice for in each case 30 minutes in an ultrasonic bath. The supernatants were filtered off, combined and evaporated to dryness in vacuo on a rotary evaporator. The residues were redissolved in 100 ml of water:ethyl acetate (EtOAc) 1:1. The organic ethyl acetate phases were separated off, dried over anhydrous sodium sulfate and evaporated to dryness on a rotary evaporator. The aqueous phase was treated twice more with in each case 50 ml of ethyl acetate, and the organic phases were separated off and combined with the first phase. The combined organic phases of the extraction gave approximately 300 mg of an oily crude product, which were dissolved in 2 ml of MeOH and filtered through a Baker (Deventer, The Netherlands) Bond Elut C18 1 ml solid-phase extraction cartridge. The filtrate was employed directly in the preparative HPLC.

Preparative HPLC was performed at 22° C. The system used, from Gilson Abimed (Ratingen, Germany), consisted of Gilson Unipoint Software, a 306 binary pump system, a 205 fraction collector, a 119 UV-Vis detector, an 806 manometric module and an 811C dynamic mixer. A Merck (Darmstadt, Germany) LichroSorb RP18 column (particle size 7 μm; column dimensions 250×25 mm) was used as stationary phase. The mobile phase used was a gradient from 0.1% aqueous trifluoroacetic acid to acetonitrile (ACN) with a continuous flow rate of 10 ml/minutes under the following elution profile: linear gradient from 20% to 50% ACN in 35 minutes; thereafter isocratic conditions at 50% ACN for 25 minutes, followed by the linear gradient from 50% ACN to 100% ACN in 40 minutes. 10 ml aliquots were fractionated and combined according to UV adsorption at 210 nm. Retention times (Rt) were 77-82 minutes for compound (2) and 91-97 minutes for compound (1).

### Example 6B

Extraction and Isolation of Compound (1) from Stirred Fermenter Cultures (10 Liter Scale)

The mycelia of fermentations of strain B. simplex DSM 14028 on the 10 liter scale were separated from the supernatant by centrifugation (10 minutes at 1000×g) and extracted directly with 2×200 ml of methanol. The methanolic extracts were evaporated to dryness and gave an oily intermediate. This product was processed as described hereinbelow by flash chromatography on silica gel material.

The culture supernatant obtained after centrifugation was applied to a 6×18 cm Lewapol (500 ml) OC 1064 (Bayer A G, Leverkusen, Germany) adsorber resin column. This column was washed with 1 l of water and eluted in succession with 2 1 of 70% methanol and 1 l of acetone. The organic eluates were concentrated to an aqueous residue on a rotary evaporator. Most of the product was recovered in the acetone fraction, which was subsequently processed via flash chromatography as described hereinbelow.

To this end, the oily intermediates of the extractions of mycelia and supernatants were dissolved in as little as possible tert-butyl methyl ether (TMBE) and bound to 400 ml of Silica Gel 60 (0.040-0.063 mm, Merck Darmstadt, Germany). The TMBE was evaporated carefully on a rotary evaporator. The intermediates which were bound to the carrier material (dry, 100 ml of silica gel) were subsequently packed into a 500 ml SIM module (Biotage). Isocratic elution was performed with 2 1 of cyclohexane (CH) followed by 1.6 l of a linear gradient from cyclohexane to TBME.

The fractions eluted with CH:TBME contained the highly concentrated product in addition to remainders of unreacted aglycon and other constituents. These fractions were concentrated on a rotary evaporator and subjected to preparative HPLC in order to isolate compound (1).

Compound (1) was isolated using the preparative HPLC system described in Example 6a using a Kromasil 100 C8 (7 μm, 250×40 mm) column from MZ Analysentechnik as stationary phase and 0.1% trifluoroacetic acid: acetonitrile (CAN) as mobile phase at a continuous flow rate of 10 ml/minute in the following gradients: 30% ACN for 20 minutes, followed by linear gradients (30%-50% ACN in 60 minutes), then again linear gradient from 50% ACN to 100% ACN in 42 minutes.

10 ml aliquots were fractionated and subsequently combined in accordance with UV absorption at 210 nm. Retention times (Rt) were 105-111 minutes for compound (2) and 115-119 minutes for compound (1).

Work-up and isolation from fermentation on a larger scale were carried out analogously to this process, extraction volumes, elution volumes and column bed volumes being increased as appropriate (i.e. matched to suit the volumes of the production cultures) and, if appropriate, intermediates being divided into aliquots before being subjected to the final HPLC separation.

### Example 7

Structure Elucidation of Compound (1)

The structure of compound (1) was elucidated with the aid of 1D and 2D nuclear resonance spectroscopy (NMR) and positive electrospray mass spectrometry (+ESI-MS). NMR spectra were recorded using a Bruker DMX500 spectrometer at 302 K in DMSO. MS spectra were recorded using an LCT ESI-TOF apparatus from Micromass.

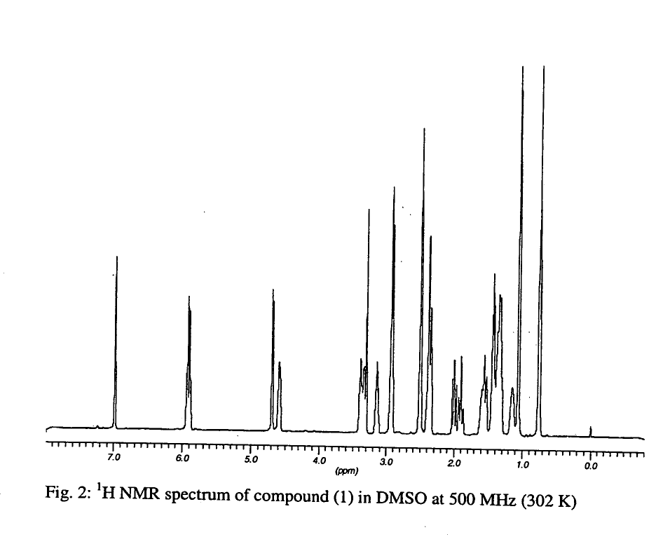

The mass as determined by (ES+I)-MS was 400 (C₂₄H₃₂O₅) (corresponding to a recorded molar mass of 401 (m/z+H)). The mass as determined by MS high-resolution (ESI+) was 401.2340 (calculated: 401.2328). The NMR data are compiled in the table which follows, and the proton spectrum is shown in FIG. 2.

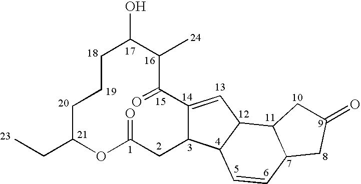

| Atom No. | C (ppm) | H (ppm) |
|---|---|---|
| 1 | 172.2 | — |
| 2 | 33.6 | 2.51; 2.93 |
| 3 | 48.7 | 2.94 |
| 4 | 40.6 | 3.39 |
| 5 | 129.1 | 5.91 |
| 6 | 129.0 | 5.94 |
| 7 | 39.9 | 2.38 |
| 8 | 43.1 | 1.91; 2.38 |
| 9 | 219.0 | — |
| 10 | 42.7 | 2.00; 2.34 |
| 11 | 44.0 | 1.35 |
| 12 | 47.9 | 2.93 |
| 13 | 147.1 | 7.00 |
| 14 | 144.2 | — |
| 15 | 203.6 | — |
| 16 | 47.8 | 3.14 |
| 17 | 70.9 | 3.35 |
| 18 | 34.9 | 1.37; 1.34 |
| 19 | 21.7 | 1.15; 1.59 |
| 20 | 29.5 | 1.54; 1.34 |
| 21 | 76.3 | 4.60 |
| 22 | 28.1 | 1.44 |
| 23 | 9.2 | 0.76 |
| 24 | 15.9 | 1.07 |
| 25 | | 3.74 (OH) |

¹H ¹³C chemical shifts of compound (1) in DMSO-d₆ measured at 302 K

The optical rotation was determined in a Perkin-Elmer Polarimeter Model 341 at 20° C. and 589 nm (length of the cuvette: 10 cm, solvent: methanol). The specific rotation of compound (1): (NaD, 589 nm) in MeOH was: −272.2°.

### Example 9

Characterization of the Strains Used

a) Streptomyces argillaceus DSM 14030:

This strain was deposited as mithramycin producer and as type strain of the new Streptomyces species S. argillaceus at the American Type Culture Collection, deposit number ATCC 12956, by Pfizer Inc., New York, USA (S. argillaceus ATCC 12956). The strain description can be found in U.S. Pat. No. 3,646,194. The culture was also deposited on O₂.08.2001 at the Deutsche Sammlung von Mikroorganismen und Zellkulturen GmbH [German Collection of Microorganisms and Cell Cultures GmbH] (DSMZ), Mascheroder Weg 1b, D-38124 Brunswick, Germany, deposit number DSM 14030, in compliance with the provisions of the Budapest treaty.

b) Streptomyces Scabies (BS 2134):

Strain BS 2134 was isolated from a soil sample collected in Rhineland-Palatinate (Germany). The culture was also deposited on 02.08.2001 at the Deutsche Sammlung von Mikroorganismen und Zellkulturen GmbH [German Collection of Microorganisms and Cell Cultures GmbH] (DSMZ), Mascheroder Weg 1b, D-38124 Brunswick, Germany, deposit number DSM 14029, in compliance with the provisions of the Budapest treaty.

Strain BS 2134 was classified as Streptomyces scabies. The results are based on a comparison with Streptomyces scabies DSM 40478 using the methods of the International Streptomyces Project (ISP) for the characterization and identification of Streptomyces species (Küster E. (1972), Int. J. Syst. Bacteriology, 22: 139), and with the aid of sequence analyses of the 16S rDNA (Maidak et. al. (1996), Nucleic Acids Res., 24: 82). The results are compiled in the table which follows. According to 16S rDNA sequence analysis, the strain shows 100% similarity with S. annulatus, which is assigned to the same species cluster as S. scabies. However, since the morphological and physiological traits in accordance with the ISP description match S. scabies better, the strain was identified as S. scabies.

ISP description. Sporophore morphology: rectiflexible, short chains with not more than 10 spores. The aerial mycelium belongs to the “gray” series, the reverse mycelium is yellow to grayish-green. No melamine pigments are formed. Sucrose, inositol and raffinose are not utilized.

c) Streptomyces spec. (BA 312):

Strain BA 312 was isolated from a soil sample collected in Rhineland-Palatinate (Germany). The culture was also deposited on 02.27.2001 at the Deutsche Sammlung von Mikroorganismen und Zellkulturen GmbH [German Collection of Microorganisms and Cell Cultures GmbH] (DSMZ), Mascheroder Weg 1b, D-38124 Brunswick, Germany, deposit number DSM 14077, in compliance with the provisions of the Budapest treaty.

It was impossible to assign strain BA 312 to a known Streptomyces species in terms of molecular biology or chemotaxonomy. The physiological test results (sugar utilization, see table hereinbelow) show a similarity with S. ochraceiscleroticus, but the morphological traits (gray aerial mycelium, rectiflexible spore chains, yellow pigmentation of the substrate mycelium, no melanin formation) and the 16S rDNA analysis do not agree with S. ochraceiscleroticus, so that the strain will have to be classified as S. spec. (see table which follows).

d) Streptomyces mirabilis (BA 579):

Strain BA 579 was isolated from a soil sample collected in Rhineland-Palatinate (Germany). The culture was also deposited on 02.27.2001 at the Deutsche Sammlung von Mikroorganismen und Zellkulturen GmbH [German Collection of Microorganisms and Cell Cultures GmbH] (DSMZ), Mascheroder Weg 1b, D-38124 Brunswick, Germany, deposit number DSM 14078, in compliance with the provisions of the Budapest treaty.

Owing to the high 16S rDNA sequence similarity and the morphological traits, this strain was classified as S. mirabilis. However, the strain differs in terms of ISP sugar utilization in as far as it shows growth with sucrose and raffinose, and in the absence of the formation of melanin (see table which follows).

ISP description: gray aerial mycelium, sporophore morphology: section Spirales, weak sporulation. The reverse mycelium is grayish-yellow in color, turning olive brown to brown. Melanin pigments are formed in peptone-yeast-iron agar. Sucrose and raffinose are not utilized.

e) Streptomyces pseudovenecuelae (WS 2199):

Strain WS 2199 was isolated from aqueous sample (Germany). The culture was also deposited on 02.27.2001 at the Deutsche Sammlung von Mikroorganismen und Zellkulturen GmbH [German Collection of Microorganisms and Cell Cultures GmbH] (DSMZ), Mascheroder Weg 1b, D-38124 Brunswick, Germany, deposit number DSM 14079, in compliance with the provisions of the Budapest treaty.

Owing to the high 16S rDNA sequence similarity, the morphological traits and the physiological test results, this strain was classified as S. pseudovenecuelae. The exception is the aerial mycelium color, which is not reddish gray, but simply gray (see the table which follows).

ISP description: aerial mycelium color: “red” series, gray has also been observed; sporophore morphology: section Rectiflexibiles. Sporophores very long (up to 50 spores). Melanin formation. D-glucose, L-arabinose, sucrose, D-xylose, inositol, D-mannitol, fructose, rhamnose and raffinose are all utilized.

| | DSM | Aerial mycelium | Reverse mycelium | | Sporophore | Melanoid | | 16S rDNA sequence |
|---|---|---|---|---|---|---|---|---|
| Strain No. | No. | color | color | Soluble pigments | morphology | pigments | DAP | similarity to type strain* |
| BS 2134 | 14029 | Gray | Grayish-yellow | None | Rectiflexibiles (RF) | None | LL- | S. annulatus 100% |
| BA 312 | 14077 | Gray | Yellow | None | Rectiflexibiles (RF) | None | LL- | S. tauricus 99.1% |
| BA 579 | 14078 | Gray | Beige | None | Spiralis | None | LL- | S. mirabilis 99.7% |
| WS 2199 | 14079 | Gray | Brownish-red | none | Rectiflexibiles (RF) | Positive | LL- | S. pseudovenecuelae 100% |

| | | | | |
|---|---|---|---|---|
| | | | | Similarity to |
| Strain- | DSM | Sugar utilization in accordance with ISP | Physiological | Reference |

| | | | | | | | | | | | | |
|---|---|---|---|---|---|---|---|---|---|---|---|---|
| No. | No. | Glucose | Arabinose | Xylose | Mannose | Fructose | Sucrose | Inositol | Rhamnose | Raffinose | groups | strains** |

| | | | | | | | | | | | | |
|---|---|---|---|---|---|---|---|---|---|---|---|---|
| BS 2134 | 14029 | + | + | + | + | + | − | + | + | − | S. violaceus | 96.9% |
| BA 312 | 14077 | + | + | + | + | + | + | + | − | + | S. ochracei- | 100% |
| | | | | | | | | | | | scleroticus | |
| BA 579 | 14078 | + | + | + | + | + | + | + | − | + | S. violaceus | 85.5% |
| WS 2199 | 14079 | + | + | + | + | + | + | + | − | + | | 99.7% |

Physiological characterization of BS 2134, BA 312, BA 579, WS 2199
**Members of one taxon have a value of >99%

f) Bacillus simplex DSM 14028:

Bacillus simplex DSM 14028 was incorporated into the strain collection Collection of Nathan R. Smith, U.S. Department of Agriculture, Washington, D.C., USA, as Bacillus megaterium NRS 610 and described newly by Priest et al. as Bacillus simplex. It was incorporated into the collection of the Deutsche Sammlung für Mikroorganismen as DSM 1318 (Priest, F. G., Goodfellow, M., Todd, C., A numerical classification of the genus Bacillus. J. Gen. Microbiol. 134: 1847-1882, 1988). The strain was redeposited on 02.08.2001 at the Deutsche Sammlung von Mikroorganismen und Zellkulturen GmbH [German Collection of Microorganisms and Cell Cultures] (DSMZ), Mascheroder Weg 1b, D-38124 Brunswick, Germany, in compliance with the provisions of the Budapest treaty, deposit number DSM 14028.

g) Bacillus megaterium DSM 333:

Bacillus megaterium DSM 333 was deposited by D. Claus at the Deutsche Sammlung für Mikroorganismen [German Collection of Microorganisms], deposit number DSM 333, and described by Hunger et al. (Hunger, W., Claus, D.: Taxonomic studies on Bacillus megaterium and on agarolytic Bacillus strains, pp. 217-239, In Berkeley, R. C. W., Goodfellow, M (eds.) The aerobic endospore-forming bacteria: classification and identification. Academic Press, London 1981).

h) Bacillus megaterium DSM 339:

Bacillus megaterium DSM 339 was deposited at the Deutsche Sammlung für Mikroorganismen by the Institute of Microbiology, University of Göttingen, deposit number DSM 339. The strain taxonomy is described by Hunger et al. (Hunger, W., Claus, D.: Taxonomic studies on Bacillus megaterium and on agarolytic Bacillus strains, pp. 217-239, In Berkeley, R. C. W., Goodfellow, M (eds.). The aerobic endospore-forming bacteria: classification and identification. Academic Press, London 1981).

i) Zygorhynchus moelleri Vuill.—Strain WP 0796:

The strain was isolated by Dr. H.-G. Wetzstein (Bayer AG) 1994 from a soil sample taken in Germany and its morphology was characterized by Dr. P. Hofmann (DSMZ). This taxonomy was confirmed with the aid of the specialist identification achieved in K. H. Domsch et al. (1980), Compendium of soil fungi Vol. 2. ICW Verlag (where it is entered as Zorrhynchus moelleri) with the aid of a microscopic study of the culture deposited at the DSMZ. However, the currently valid generic name is given as Zygorhynchus (instead of Zygorrhynchus) according to Hawksworth, D. L. et al. (eds.): Ainsworth & Bisby's Dictionary of the Fungi, CAB International, London, 8^{th} edition (1996). The culture was also deposited on 03.21.2001 at the Deutsche Sammlung von Mikroorganismen und Zellkulturen GmbH [German Collection of Microorganisms and Cell Cultures GmbH] (DSMZ), Mascheroder Weg 1b, D-38124 Brunswick, Germany, deposit number DSM 14198, in compliance with the provisions of the Budapest treaty.

j) Mucor circinelloides Van Tiegh—Strain WP 0799:

The strain was isolated by Dr. H.-G. Wetzstein (Bayer AG) 1994 from a soil sample taken in Germany and its morphology was characterized by Dr. P. Hofmann (DSMZ). This taxonomy was confirmed with the aid of the specialist identification achieved in K. H. Domsch et al. (1980), Compendium of soil fungi Vol. 2. ICW Verlag (where it is entered as Mucor circillenoides) with the aid of a microscopic study of the culture deposited at the DSMZ. The culture was also deposited on 03.21.2001 at the Deutsche Sammlung von Mikroorganismen und Zellkulturen GmbH [German Collection of Microorganisms and Cell Cultures GmbH] (DSMZ), Mascheroder Weg 1b, D-38124 Brunswick, Germany, deposit number DSM 14199, in compliance with the provisions of the Budapest treaty.

### Example 10

Chemical Synthesis of Compound (1) by Oxidation with Pyridinium Dichromate

Compound (1) was prepared from compound (2) by oxidation with pyridinium dichromate: 46.55 g (115.6 mmol) of compound (2) were dissolved in 1100 ml of absolute dichloromethane under inert gas and treated with 43.51 g (115.6 mmol) of pyridinium dichromate. After the mixture had been stirred for 4 hours at 25° C. and 900 ml of diethyl ether had been added, the chromium salts which had precipitated were filtered off and the filtrate was concentrated in vacuo. Column chromatography on silica gel (eluent: cyclohexane/ethyl acetate 1:1, then 100% ethyl acetate) yielded 3.68 g of 9,17-diketospinosyn aglycon and 23.40 g of an approx. 9:1 mixture of compound (2) and 17-ketospinosyn aglycon in addition to 11.74 g of recovered compound (2). Compound (1) is concentrated to >98% by recrystallization of the mixture from cyclohexane/ethyl acetate. This gives 20.78 g of compound (1) as colorless crystals.—Compound (1): DC: R_{f} (SiO₂, ethyl acetate)=0.44—¹H NMR: CDCl₃, δ=6.77 (s, 13-H); 5.97 (d, 6-H); 5.88 (m, 5-H); 4.72 (m, 21-H); 3.69 (m, 17-H) inter alia—LC/ESI-MS: m/z=401 (25%) [M]⁺, 289 (100%). The compound (1) which has been prepared in this manner is identical in all spectroscopic data with the compound (1) which has been produced by bioconversion.—17-ketospinosyn aglycon: DC: R_{f} (SiO₂, ethyl acetate)=0.40—¹H NMR: CDCl₃, δ=6.97 (s, 13-H); 5.90 (d, 6-H); 5.79 (m, 5-H); 4.85 (m, 21-H); 4.45 (m, 9-H); 4.25 (q, 16-H) inter alia—LC/ESI-MS: m/z=401 (100%) [M+H]⁺, 273 (70%).—9,17-diketospinosyn aglycon: DC: R_{f} (SiO₂, ethyl acetate)=0.64—¹H NMR: CDCl₃, δ=6.92 (s, 13-H); 5.97 (d, 6-H); 5.87 (m, 5-H); 4.85 (m, 21-H); 4.25 (q, 16-H), inter alia—LC/ESI-MS: m/z=399 (100%) [M+H]⁺.

### Example 10A

Chemical Synthesis of Compound (1) by Swern Oxidation

As an alternative, compound (1) was prepared from compound (2) by Swern oxidation (variant of Corey and Khim (1972), J. Am. Chem. Soc., 94: 7586): 133 mg (1 mmol) of N-chlorosuccinimide were suspended in 10 ml of absolute dichloromethane under inert gas and, at −70° C., treated with 66 mg (1.07 mmol) of dimethyl sulfide. After the mixture had been stirred for 30 minutes, a solution of 402 mg (1 mmol) of compound (1) in 2 ml of absolute dichloromethane was slowly added dropwise. After the reaction mixture had been stirred for 2 hours at −70° C., 132 μl (0.95 mmol) of triethylamine were added dropwise and the mixture was warmed to room temperature in the course of 16 hours. After dilution with 100 ml of dichloromethane, the mixture was washed in succession with 100 ml of water and 100 ml of saturated aqueous sodium chloride solution, dried over sodium sulfate and concentrated in vacuo. Column chromatography on silica gel (eluent: cyclohexane/ethyl acetate 2:1) yielded 246 mg of compound (1) in addition to 37 mg of 9,17-diketospinosyn aglycon and 120 mg of recovered compound (2). Compound (1) which has been prepared in this manner is identical in all spectroscopic data with compound (1) which has been prepared by oxidation with pyridinium dichromate or produced by bioconversion.

### Example 10b

Chemical Synthesis of 5,6-dihydro-9-ketospinosyn Aglycon (Compound (3))

Compound (3) was prepared from compound (4) analogously to Example 10a) by oxidation with pyridinium dichromate: 202 mg (0.5 mmol) of compound (4) were dissolved in 5 ml of absolute dichloromethane under inert gas and treated with 188 mg (0.5 mmol) of pyridinium dichromate. After the mixture had been stirred for 4 hours at 25° C. and 5 ml of diethyl ether had been added, the chromium salts which had precipitated were filtered off and the filtrate was concentrated in vacuo. Column chromatography on silica gel (eluent: cyclohexane/ethyl acetate 1:1, then 100% ethyl acetate) yielded 24 mg of 5,6-dihydro-9,17-diketospinosyn aglycon and 103 mg of compound (3) in addition to 41 mg of recovered starting material compound (4).—Compound (3): DC: R_{f} (SiO₂, ethyl acetate)=0.44—¹H NMR:CDCl₃, δ=6.83 (s, 13-H); 4.68 (m, 21-H); 3.69 (m, 17-H) inter alia—LC/ESI-MS: m/z=403 (23%) [M+H]⁺, 291 (100%).—5,6-dihydro-9,17-diketospinosyn aglycon: DC: R_{f} (SiO₂, ethyl acetate)=0.64.—¹H NMR: CDCl₃, δ=6.99 (s, 13-H); 4.82 (m, 21-H); 4.23 (q, 16-H), inter alia—LC/ESI-MS: m/z=423 (100%) [M+Na]⁺.

### Example 11

Synthesis of the 9-Ketospinosyn A-9-Pseudoaglycon

a) Synthesis of the trichloroacetimidate (cf. also the method described by G. R. Duffin et al. (2000), J. Chem. Soc., Perkin Trans. 1: 2237-2242):

200 mg (1.256 mmol) of α-D-forosamine were stirred in 10 ml of methylene chloride, treated with 419.0 mg (2.902 mmol) of trichloroacetonitrile and 108.5 mg (0.333 mmol) of cesium carbonate, and the mixture was stirred for approximately 2 hours at room temperature. The reaction mixture was subsequently diluted with methylene chloride and washed with aqueous sodium hydrogen carbonate solution, the organic phase was dried over magnesium sulfate and the solvent was stripped off in vacuo. This gives 303 mg (79.5%) of trichloroacetimidate, which can be used directly for the glycosidation reaction (b).

C₁₀H₁₇Cl₃N₂O₂ (303.6)

LC/MS: m/z=303 (100%) [M]⁺

b) Glycosidation of compound (1) with trichloroacetimidate (cf. also the method described by G. R. Duffin et al. (2000), J. Chem. Soc., Perkin Trans. 1: 2237-2242):

100 mg (0.250 mmol) of compound (1) were stirred with 2 ml of methylene chloride, and treated with 50 mg of molecular sieve 4A and 151 mg (0.500 mmol) of freshly prepared trichloroacetimidate (a). 49.4 mg (0.348 mmol) of boron trifluoride etherate were subsequently added, and the reaction mixture was stirred for approximately 18 hours at room temperature. After removal of molecular sieve 4A, the residue was diluted with a little methylene chloride and washed with aqueous sodium hydrogen carbonate solution. The organic phase was dried over magnesium sulfate and the solvent was stripped off in vacuo.

C₃₂H₄₇NO₆ (541.7)

LC/MS: m/z=542 (100%) [M]⁺ (cf. WO 97/00265 and U.S. Pat. No. 6,001,981)

We claim:

1. A compound of the formula (I) 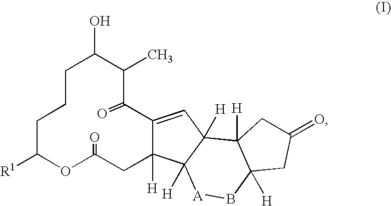
in which
R¹ represents methyl or ethyl, and
A-B represents one of the following groups: —HC═CH—, —HC═C(CH₃)—, —H₂C—CH₂—, —H₂C—CH(CH₃)—.

2. A compound as claimed in claim 1, characterized in that
R¹ represents ethyl.

3. A compound as claimed in claim 1 or 2, characterized in that
A-B represents the group —HC═CH—.

4. A process for the preparation of compounds as claimed in any of claims 1 to 3, characterized in that compounds of the general formula (II) 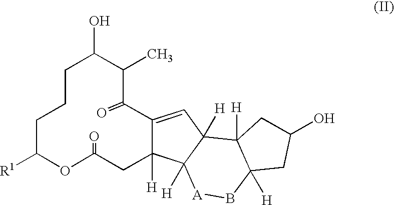
in which
R¹ and A-B have the meanings given in any of claims 1 to 3,
are reacted with an oxidant, if appropriate in the presence of a diluent.

5. A process as claimed in claim 4, characterized in that the oxidant employed is pyridium dichromate or N-halosuccinimides in the presence of dimethyl sulfide.

6. A process as claimed in claim 4 or 5, characterized in that the diluent employed is dichloromethane.

7. A process for the production of compounds as claimed in any of claims 1 to 3, characterized in that compounds of the general formula (II) 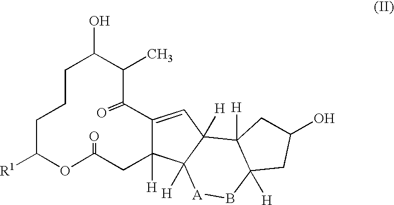
in which
R¹ and A-B have the meanings given in any of claims 1 to 3,
are brought into contact with a microorganism in an aqueous nutrient medium under aerobic conditions or with an enzyme extract prepared therefrom or with one or more enzymes isolated therefrom.

8. A process as claimed in claim 7, characterized in that the microorganism employed is a microorganism from the genus Bacillus, from the group of the Actinomycetes or from the class of the Zygomycetes.

9. A process as claimed in claim 8, characterized in that a strain of the species Bacillus megaterium or Bacillus simplex is employed.

10. A process as claimed in claim 9, characterized in that the strain has the characterizing traits of the strains Bacillus megaterium DSM 339, Bacillus megaterium DSM 333 or Bacillus simplex DSM 14028.

11. A process as claimed in claim 8, characterized in that a microorganism from the genus Streptomyces is employed as microorganism from the group of the Actinomycetes.

12. A process as claimed in claim 11, characterized in that a strain from the species Streptomyces argillaceus, Streptomyces scabies, Streptomyces mirabilis or Streptomyces pseudovenecuelae is employed as microorganism from the genus Streptomyces.

13. A process as claimed in claim 12, characterized in that the strain has the characterizing traits of the strains Streptomyces argillaceus DSM 14030, Streptomyces scabies DSM 14029, Streptomyces mirabilis DSM 14078 or Streptomyces pseudovenecuelae DSM 14079.

14. A process as claimed in claim 8, characterized in that a microorganism from the genus Zygorhynchus, in particular a strain from the species Zygorhynchus moelleri, is employed as microorganism from the class of the Zygomycetes.

15. A process as claimed in claim 14, characterized in that the strain has the characterizing traits of the strain Zygorhynchus moelleri DSM 14198.

16. A process as claimed in claim 8, characterized in that a microorganism from the genus Mucor, in particular a strain from the species Mucor circinelloides, is employed as microorganism from the class of the Zygomycetes.

17. A process as claimed in claim 16, characterized in that the strain has the characterizing trait of the strain Mucor circinelloides DSM 14199.

18. A process as claimed in any of claims 4 to 17, characterized in that the compounds as claimed in any of claims 1 to 3 are isolated.

19. The use of compounds as claimed in any of claims 1 to 3 for the preparation of Spinosyn derivatives.
